(19) United States
(12) Patent Application Publication
(10) Pub. No.: US 2015/0231306 A1
(43) Pub. Date: Aug. 20, 2015

(54) COATED MEDICAL DEVICE

(71) Applicant: Lenn R. HANN, Wheaton, IL (US)

(72) Inventor: Lenn R. HANN, Wheaton, IL (US)

(21) Appl. No.: 14/312,362

(22) Filed: Jun. 23, 2014

Related U.S. Application Data
(60) Provisional application No. 61/941,889, filed on Feb. 19, 2014.

(51) Int. Cl. A61L 29/08

(52) U.S. Cl. CPC A61L 29/085; A61L 2420/08; A61L 2420/02

(57) ABSTRACT

A method and apparatus for inserting a medical device such as a cannula tip within a peripheral vein of a human body wherein the medical device includes a micro- or nano-structured superhydrophilic basecoat and a liquid topcoat, together comprising a superhydrophobic coating, which inhibit occlusion and/or catheter related bloodstream infection.

### CROSS REFERENCE TO RELATED APPLICATION

This Patent Application claims priority to U.S. Provisional Patent Application, Ser. No. 61/941,889, filed on 19 Feb. 2014 which is hereby incorporated by reference herein in its entirety and is made a part hereof, including but not limited to those portions which specifically appear hereinafter.

### BACKGROUND OF THE INVENTION

1. Field of the Invention

This invention relates to a medical device and method, and more particular, to an intravenous apparatus having micro- or nano-structure superhydrophilic basecoat and a liquid top coat, together comprising a superhydrophobic coating.

2. Discussion of Related Art

Medical devices, including intravenous devices, are widely used for patients that require medication, fluids, and similar care applications found in hospitals and similar medical care facilities. Such devices include cannula-over-needle apparatus, in which a flexible plastic or polymer cannula comes mounted on a metal insertion needle. Once the tip of the needle and cannula are located properly in the vein, the insertion needle is withdrawn and discarded. Meanwhile, the cannula is advanced inside the vein to a predetermined position where an external hub or valve area of the catheter is secured to the patient's body by medical tape or the like to hold it in place. Blood is often withdrawn at the time of the initial insertion of the cannula into the patient's vein to confirm placement. This is the most common intravenous access method used in both hospitals and in the field by paramedics or emergency medical technicians (EMTs).

The calibers of cannula generally range from 12 to 26 gauge with 12 being the largest and 26 being the smallest. The part of the catheter remaining outside of the skin is called the IV connecting hub or IV valve that is connected to the IV lines back to the IV bag of fluids. For example, an all-purposes IV cannula for infusions and blood draws might be an 18 and 20 gauge sized cannula manufactured by BD/Becton Dickinson or B. Braun. This intravenous cannula comes with an inner needle that is removed once the flexible portion of the cannula is fully inserted into the patient's vein.

Due to varying conditions within the medical facility and/or different skill levels of medical personnel inserting the IV device, such as a cannula, into the peripheral vein of a patient's hand or arm, complications sometimes develop in a number of the patients as a result. Such complications include occlusion which is a gradual blockage of the cannula which may result from improper device insertion, placement or the body's treatment of the cannula as a wound site. In addition, catheter related bloodstream infection (CRBSI), also referred to as catheter related sepsis, may result from catheters or similar devices that include the presence of bacteraemia. These conditions may arise as a result of improper insertion or occlusion of the cannula or as a result of contaminated devices or work areas. Many serious complications can result from sterilization issues, less-than-optimum facilities, and/or improper cannula insertion into the vein. The potential complications include sepsis, edema causing tissue damage or may even include necrosis depending on the medication or fluid being infused. This extravasation is a leakage of infused fluids into the vasculature of the subcutaneous tissue surrounding the vein. The leakage of high osmotic solutions or chemotherapy fluids can result in significant tissue destruction or other complications.

### SUMMARY OF THE INVENTION

A medical device, such as a cannula with an insertion needle, preferably includes a micro- or nano-textured superhydrophilic basecoat positioned over at least one of the cannula exterior and/or the cannula interior. A liquid topcoat is preferably positioned on the basecoat, together forming a superhydrophobic coating, such hydrophobic coating inhibiting occlusion and catheter related bloodstream infection.

Additional embodiments may include modifying compounds to assist in compatibility, medicine delivery and/or other benefits.

The above summary of the present invention is not intended to describe each illustrated embodiment or every implementation of the present invention. The figures and the detailed description which follow more particularly exemplify these embodiments.

### BRIEF DESCRIPTION OF THE DRAWINGS

This invention is explained in greater detail below in view of exemplary embodiments shown in the drawings, wherein:

FIG. 1 shows a schematic of a cannula in accordance with one embodiment of the present invention;

FIG. 2 shows a schematic of a cannula in accordance with one preferred embodiment of the present invention;

FIG. 3 shows a schematic of a cannula in accordance with one preferred embodiment of the present invention; and

FIG. 4 shows a schematic of a cannula in accordance with one preferred embodiment of the present invention.

### DESCRIPTION OF PREFERRED EMBODIMENTS

The present invention relates to an intravenous apparatus and method for proper insertion of a cannula tip of a catheter into a vein for infusion of intravenous (IV) fluids.

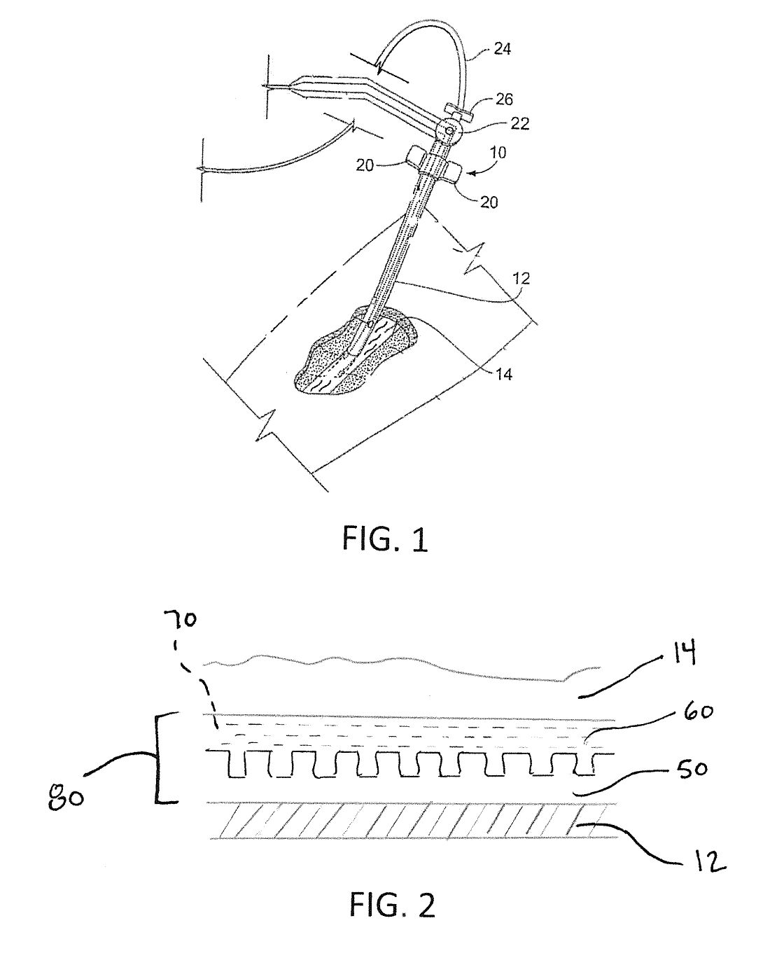

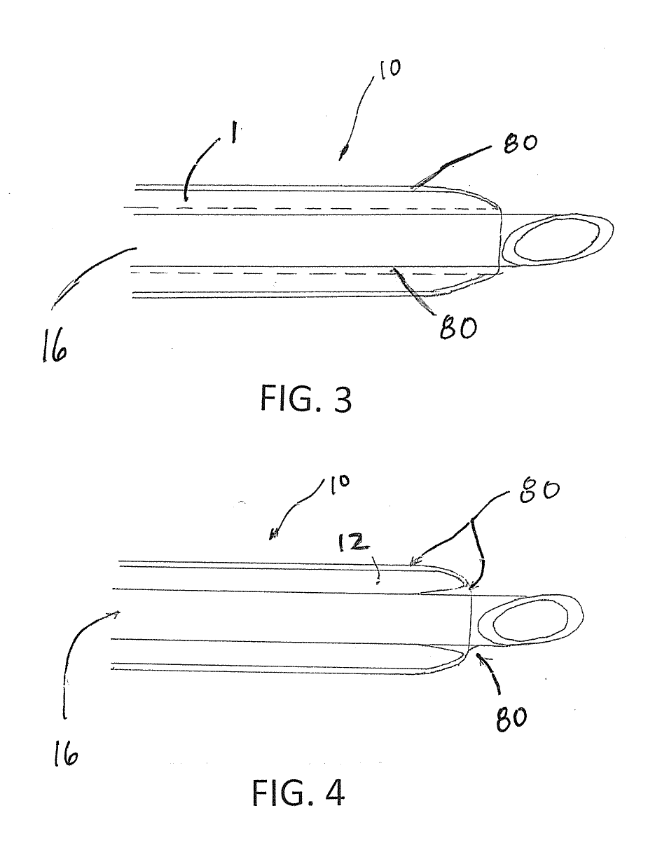

As mentioned above, one embodiment is illustrated in FIG. 1 and includes a catheter 10 constructed in accordance with the present invention. The typical catheter 10 includes a short polymer tube (a few centimeters long) inserted through the skin into a peripheral vein 14 (any vein generally not inside the chest or abdomen). This is usually in the form of a flexible cannula 12 over-needle device, in which a flexible plastic cannula 12 comes mounted on a metal insertion needle (insertion needle 16 not shown in FIG. 1 as already withdrawn from cannula 12, see FIGS. 3 and 4). Once the tip of the needle and cannula 12 are located within the vein 14 the insertion needle is withdrawn and discarded. The cannula 12 is further advanced inside the vein to an appropriate position and then secured with medical tape or the like over a pair of plastic wings 20 secured to the tubing near a port or hub 22. An IV line 24 may further connect to the port or hub 22 through a male fluid input 26 that is inserted into the IV line 24.

According to one preferred embodiment of this invention, the smart IV cannula 12 as shown and described may further include a superhydrophobic coating and/or construction. Accordingly, the cannula 12 may include at least one of: a micro- or nano-coating over and/or within the cannula 12; the cannula 12 may be constructed of urethane, Teflon® or silicone; and/or a hybrid construction, such as silicone oil impregnated urethane.

FIG. 2 shows a schematic of one embodiment of the invention wherein the cannula 12, such as a urethane cannula, includes a micro or nano-textured basecoat 50, over which a liquid topcoat 60 is applied. The basecoat 50 and liquid topcoat 60 are together referred to as the superhydrophobic coating 80. “Superhydrophobic” may be quantified as a coating wherein the contact angles of a water droplet exceed 150° and the roll-off angle/contact angle hysteresis is less than 10°. This is also referred to as the “Lotus Effect” after the superhydrophobic leaves of the lotus plant. As described herein, the liquid topcoat 60 may be further modified to include one or more modifying compounds.

According to one preferred embodiment of this invention, a medical device such as an IV cannula, such as shown and described in U.S. Ser. No. 14/168,902, may include a superhydrophobic coating and/or construction. Accordingly, the cannula may include at least one of: a micro- or nano-structure basecoat 50 over and/or within the cannula 12; a superhydrophic topcoat 60 over the basecoat; the cannula 12 may be constructed of urethane, Teflon® or silicone; and/or a hybrid construction, such as silicone oil impregnated urethane.

A suitable basecoat 50 and/or topcoat 60 may comprise a coating such as described in U.S. Pat. Nos. 8,574,704 and 8,535,779 to Smith et al., which are hereby incorporated by reference. The Smith et al. patents describe non-wetting surfaces that include a liquid impregnated within a matrix of micro/nano-engineered features on the surface, or a liquid filling pores or other tiny wells on the surface. Such a product, called Liquiglide™, may be used to coat the cannula described herein. As described, a micro/nano-engineered surface coating enables a durable liquid-impregnated surface coating to be placed over the full exterior and interior surfaces of an IV cannula.

Alternatively, or in addition, the cannula 12 may be constructed of a non-stick material such as Teflon® or silicone. The cannula 12 may be wholly constructed of such material or partially constructed, coated and/or reinforced with such material.

According to another alternative of the subject invention, the cannula 12 may comprise a hybrid construction that includes a hydrophobic coating impregnated within or coated over and around a non-stick construction. Such, hybrid construction may include a silicone oil impregnated urethane construction.

A benefit of such liquid-impregnated surface coating or alternatively described constructions is to inhibit the initial “seed” adhesion of blood protein fibrin to the cannula surface, thus preventing further fibrin accretion at the orifice of the tip of the cannula, thus preventing IV cannula occlusion.

The subject invention may be preferably utilized in connection with micro- or nano-scale coatings, such as described in U.S. Pat. Nos. 8,574,704 and 8,535,779 to Smith et al., to include application to several additional medical devices as follows: (1) peripheral IV cannulas 12, as described above; (2) implanted port catheters (“portacath”); (3) peripherally inserted central catheters (“PICC”); and/or (4) subcutaneous cannulas used with wearable insulin and chemotherapy pumps. In such devices, the superhydrophobic coating is preferably applied to the cannula to prevent occlusion or catheter related bloodstream infections (CRBSIs).

The subject invention may be further or alternatively utilized in hemodialysis fistulas, specifically prosthetic hemodialysis access arteriovenous grafts (AVGs). In this application, the superhydrophobic topcoat 60 is preferably applied on cannula tips and within the fistula to prevent clotting.

The subject invention may be further or alternatively utilized in surgical drains used to evacuate body fluids generated during post-surgical wound healing. In this application, the superhydrophobic topcoat 60 is preferably applied at the tip of the drain and within to prevent clotting.

The subject invention may be further or alternatively utilized in stents used in vascular surgery to prevent blood coagulation, as well as other implanted stents that may benefit from a non-wetting superhydrophobic topcoat 60. Such stents include ureteral, urethral, biliary, duodenal, colonic, and pancreatic stents. In these applications, the superhydrophobic topcoat 60 is preferably applied over the entire area of the stent to prevent clotting, tissue adhesion, and other fluid adhesions.

Each of the described medical devices is subject to unwanted blood coagulation during normal use and operation. Significant savings in cost, infection risk, and patient discomfort can be made by adding micro- or nano-structure superhydrophilic basecoats 50 and/or superhydrophobic topcoats 60 to these devices.

According to one preferred embodiment, IV cannula coating clearance may be accommodated. The application of a liquid-containing superhydrophobic topcoat 60 to an IV cannula 12 preferably accounts for the thickness of the basecoat 50 and/or the topcoat 60 and utilizes a thinner cannula 12, a thinner hollow needle, and/or a larger diameter cannula 12 so that there is room for the interior basecoat 50 and/or topcoat 60.

According to one preferred embodiment, the urethane cannula surface is prepared for optimum basecoat 50 adhesion. Methods of doing this may include: plasma ion treatment, heat and vacuum or some combination of these three.

Further, the shelf life of one or more components of the subject invention may be of concern. For instance, the lifespan of a superhydrophopic liquid topcoat 60 once applied to a urethane catheter 12, then sterilized and packaged should be accommodated. Basecoats 50 and/or superhydrophobic topcoats 60 according to this invention preferably utilize FDA approved compounds to build their coatings, including starches and waxes, including beeswax, for the basecoat 50, and water, food-grade oils, including mineral, palm and citrus oils, and silicone oils for the superhydrophobic topcoat 60. A preferred combination permits FDA approval in human IV use while also providing acceptable shelf life prior to use. A starch/beeswax basecoat 50 and a mineral or silicone oil top coat 60 should provide an adequate balance between FDA approval and acceptable shelf life.

An additional solution to improve shelf life according to one embodiment of the invention is wet storage. In this embodiment, the cannula assembly may be stored in a liquid-filled package. The liquid would preferably be similar or identical to the liquid top coat 60 of the superhydrophobic coating 80 of the cannula 12. Such storage method would inhibit liquid loss due to evaporation, osmosis or other packaging porosity effects.

According to one preferred embodiment, a coated cannula 12 should be sterilized for packaging. As described above, such packaging should preferably have suitable shelf life for potentially years of storage prior to use. Preferred methods for this task include ionizing radiation, either gamma ray or electron beam. Alternative, or in addition, gas treatment, either ethylene oxide or formaldehyde may be utilized in connection with improving shelf life. However, this method must include safeguards against gas impingement or absorption into the liquid surface coat. Alternatively, or in addition, autoclave heat treatment may be used provided it does not damage the structure of any FDA-approved starch/wax basecoat. Alternatively, or in addition, an aseptic assembly and packaging may be utilized.

Another embodiment of the present invention may include utilization of the superhydrophobic topcoat 60 to include modifying compounds 70 such as drugs, chemical compounds or other substances for conveyance into the vein for the duration of IV use. The nature of the liquid and the drug concentration will determine the release rate.

In one example, the superhydrophobic topcoat 60 may include a modifying compound 70 comprising an amount of the drug Alteplase (Cathflo Activase) to aid in preventing catheter occlusion from fibrin adhesion. Another preferred drug for this purpose may be Drotrecogin alfa (Xigris) which also aids in preventing sepsis. In another example, the liquid topcoat 60 may contain a modifying compound 70 comprising antibiotic, anti-sepsis or anti-inflammatory drugs, or any combination thereof.

According to another embodiment of the present invention, a specific makeup of superhydrophic topcoat 60 is tailored for specific applications. Specifically, one objective is to increase biocompatibility between the medical device (stent, portacath, any long-term implanted device) and the human host. To accomplish this, the topcoat 60 may be created or augmented using components of the patients own blood. The most likely candidates to improve biocompatibility are the patient's plasma and the patient's platelet rich plasma, PRP, which is extracted after a centrifuge process. Testing should show that a topcoat 60 including the patient's blood components will increase biocompatibility and reduce inflammation, clotting, and immune responses. This custom-tailored liquid top coat may be combined with one or more modifying compounds as described above to carry drugs or other chemical compounds. Specifically, a saline-based liquid topcoat 60 may be preferable for this application likely using a body fluid (plasma, PRP) liquid topcoat. PRP may contain a number of biological growth and healing factors, such as platelet-derived growth factor; transforming growth factor beta; fibroblast growth factor; insulin-like growth factor 1 or 2; vascular endothelial growth factor; epidermal growth factor; Interleukin 8; keratinocyte growth factor; and/or connective tissue growth factor.

According to one preferred embodiment of the invention, the basecoat 50 may utilize a hardened beeswax to create a “self-healing” property to the superhydrophobic topcoat 60. One objective of this embodiment is to engineer a basecoat 50 that resists internal body degradation and seeks to bind with body fluids. This would of course prove advantageous for an implanted medical device and could lead to greater biocompatibility and tissue integration. This embodiment may be further useful for a stent or IVG used for dialysis.

As described above, a preferred method of manufacture of the subject medical device includes forming a cannula 12 from at least one of TEFLON, urethane and silicone; coating the cannula 12 with a micro- or nano-textured basecoat 50; and coating the basecoat 50 with a liquid topcoat 60 positioned on the basecoat 50, the basecoat 50 and topcoat 60 together forming a superhydrophobic coating 80.

In addition, multiple topcoat formulations may be utilized depending on where the coating occurs. For instance, a first topcoat may be positioned within an interior of the cannula 12 and a second topcoat, having different properties from the first topcoat, may be positioned on an exterior of cannula 12.

Conventional assembly techniques for urethane IV catheter and insertion needle are established and inexpensive. However, as described above, such techniques suffers the significant problems of occlusion and catheter related blood stream infections (CRBSIs). One objective of the present invention is to eliminate occlusion and CRBSIs through the use of superhydrophobic coatings, including superhydrophobic coatings modified to contain chemicals, drugs, body fluids and modified body fluids.

The subject invention may be manufactured using one of several methods. For instance, full-length internal coating, is possible where clearance between the metal insertion needle 16 (as shown in FIGS. 3 and 4) and the catheter 10 is feasible. In such a method, a full-length coating (of one or both of the basecoat 50 and the topcoat 60) may be applied to the interior of the cannula 12. This coating may differ between the external (blood stream contact) coating and the internal (metal insertion needle to urethane cannula) coating. The internal coating might be engineered to resist sticking, friction and compression. The internal coating may be further engineered to resist shearing off or other damage during assembly with the metal insertion needle. Such internal coating may be a more silicone or oil based formulation.

Another possible method of manufacture involves partial, orifice only internal coating. The cannula 12 in this method may be designed to include a flared orifice at the tip, such as shown in FIG. 4. Accordingly, the insertion needle 16 may be assembled in a conventional manner and the exterior of the cannula 12 may be coated as well as the interior of the flared portion of the cannula. In this manner, superhydrophobic coatings are placed where needed to prevent occlusion/CRBSIs and leave the remainder of the cannula interior uncoated as per current practice. In this embodiment, the coating may be sprayed on after insertion needle 16 is assembled into the catheter 10. This may leave a superhydrophobic coating “fillet” at the orifice that may be useful in preventing occlusion. Once the insertion needle is withdrawn, the liquid top coat fillet may retract to form a liquid torus shape at the orifice of the catheter.

Also note that the infusate might become mixed or partially mixed with the liquid topcoat 60 of the superhydrophic coating 80. In such event, the liquid topcoat may be applied to minimize mixing with the infusate.

Another embodiment of this invention may address problems related to damage to the superhydrophobic coating 80 that manifest when the cannula 12 is inserted through the skin of the patient. The coating 80 may be compressed, thinned or sheared during passage through the skin or vein. To mitigate such risk, one solution is to engineer the liquid topcoat 60 to resist the damage through the addition of human compatible gelling or thickening agents to the liquid topcoat 60. Such gelling agents may permit the coating 80 to retain superhydrophic properties but would be toughened to increase insertion durability and overall reliability during the term of use within the patient.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

What is claimed is:

1. A medical device for minimizing a risk of occlusion and catheter related bloodstream infection in a vicinity of an injection site, the medical device comprising:
a cannula;
an insertion needle at a distal end of the cannula;
a micro- or nano-textured basecoat positioned over at least one of an exterior and interior of the cannula; and
a liquid topcoat positioned on the basecoat, such topcoat inhibiting occlusion and catheter related bloodstream infection.

2. The medical device of claim 1 wherein the basecoat comprises a substrate of micro- or nano-engineered features.

3. The medical device of claim 1 wherein the cannula is constructed of at least one of TEFLON, urethane and silicone.

4. The medical device of claim 1 wherein the cannula includes a pretreated surface for coating adhesion, the pretreated surface comprising at least one of plasma ion treatment, heat and vacuum.

5. The medical device of claim 1 further comprising a modifying compound within the topcoat, wherein the modifying compound comprises at least one of chemical compounds, drugs, human fluids.

6. The medical device of claim 2 wherein the substrate comprises at least one of starch, beeswax, vegetable waxes, and petroleum-based waxes.

7. The medical device of claim 5 wherein the modifying compound comprises a drug for delivery into the injection site during use of the medical device.

8. The medical device of claim 7 wherein the drug comprises at least one of Alteplase and Drotrecogin alfa.

9. The medical device of 7 wherein the drug comprises at least one of an antibiotic, anti-sepsis and anti-inflammatory.

10. The medical device of claim 5 wherein the modifying compound comprises a composition including blood from a patient that will receive the medical device.

11. The medical device of claim 5 wherein the modifying compound comprises a composition including at least one of a body fluid and a processed body fluid from a patient to receive the medical device.

12. The medical device of claim 5 wherein the modifying compound comprises human plasma.

13. The medical device of claim 1 further comprising a composition including a drug and a body fluid from a patient that will receive the medical device.

14. The medical device of claim 1 wherein the liquid topcoat comprises at least one of mineral oil, saline, glycol, polyvinyl alcohol, glycene and silicone oil.

15. The medical device of claim 1 wherein the liquid topcoat further comprises a human compatible gelling or thickening agent.

16. A method of manufacturing a medical device comprising:
forming a cannula from at least one of TEFLON, urethane and silicone;
coating the cannula with a micro- or nano-textured basecoat; and
coating the basecoat with a liquid topcoat positioned on the basecoat, together forming a superhydrophobic coating.

17. The method of manufacturing of claim 16 further comprising:
applying a modified liquid topcoat over the basecoat.

18. The method of manufacturing of claim 17 further comprising:
integrating a patient's body fluid into the modified liquid topcoat.

19. The method of claim 16 wherein a first topcoat is positioned within an interior of the cannula and a second topcoat, having different properties from the first topcoat, is positioned on an exterior of cannula.

20. The method of claim 16 further comprising:
integrating a human compatible gelling or thickening agents with the liquid topcoat.
